(19) United States
(12) Patent Application Publication
(10) Pub. No.: US 2004/0063692 A1
(43) Pub. Date: Apr. 1, 2004

(54) Bazedoxifene treatment regimens

(75) Inventors: Barry Samuel Komm, Wayne, PA (US); Simon Nicholas Jenkins, Audubon, PA (US); James Charles Ermer, Eagleville, PA (US); Mark Andrew Collins, Schwenksville, PA (US); Geraldine Marie Ferron, Wayne, PA (US); Pol Boudes, Berwyn, PA (US); Wendy Ann Dulin, Tuxedo, NY (US)

Correspondence Address: COZEN O' CONNOR, P. C., 1900 MARKET STREET, PHILADELPHIA, PA, 19103-3508, US

(73) Assignee: Wyeth, Madison, NJ

(21) Appl. No.: 10/460,843

(22) Filed: Jun. 13, 2003

Related U.S. Application Data
(60) Provisional application No. 60/388,596, filed on Jun. 13, 2002.

(51) Int. Cl. A61K 31/55

(52) U.S. Cl. 514/217.09

(57) ABSTRACT

This invention relates to extended dosing regimens for the selective estrogen receptor modulator bazedoxefine (1-[4-(2-azepan-1-yl-ethoxy)-benzyl]-2-(4-hydroxy-phenyl)-3-methyl-1 H-indol-5-ol).

### CROSS-REFERENCE TO RELATED APPLICATIONS

This application claims priority benefit of U.S. Provisional Application Serial No. 60/388,596 filed Jun. 13, 2002, which is hereby incorporated by reference in its entirety.

### BACKGROUND

This invention relates to extended dosing regimens for the selective estrogen receptor modulator bazedoxefine (1-[4-(2-azepan-1-yl-ethoxy)-benzyl]-2-(4-hydroxy-phenyl)-3-methyl-1H-indol-5-ol).

Classically, estrogens are known as sex steroids affecting the reproductive tract that are required for the development of secondary sexual characteristics. However, estrogens are no longer regarded as strictly reproductive hormones and the appreciation of their effect on many organ systems has grown considerably in recent years. For example, although hormone replacement therapy was first developed to relieve menopausal hot flushes, its indications have expanded to include the treatment of vaginal atrophy and prevention of osteoporosis. Partly because such large numbers of women have used replacement therapy, a sizable body of evidence has accumulated strongly suggesting that estrogens have beneficial effects on many other organs including the bladder (improving tone and reducing incontinence), colon (reduction of cancer risk), brain (improved cognition) and cardiovascular system (improved lipid profile, reduction of risk of disease).

Estrogens can exert their effects on cells in several ways, and the most well-characterized mechanism of action is via their interaction with receptors (ERs), leading to alterations in gene transcription. To date, two ERs have been discovered: ERα and ERβ. ERs are ligand-activated transcription factors and belong to the nuclear hormone receptor superfamily. Other members of this family include the progesterone, androgen, glucocorticoid and mineralocorticoid receptors.

The development of therapeutically useful and selective ER ligands has become possible, not only because of our increased understanding of the complexities of estrogen biology, but also because a wide variety of ER ligands have been developed over the years, some of which exhibit unexpected activities. ERs have a relatively large and flexible binding pocket [Anstead, G. M., Steroids 1997, 62: 268-303]. which can accommodate structurally diverse ligands. These ligands include steroids, (e.g. 17β-estradiol, estrone), phytoestrogens (e.g. genistein, coumestrol), and xenobiotics (e.g. polychlorinated biphenols). Tradionally, compounds having roughly the same biological effects as 17β-estradiol, the most potent endogenous estrogen, are referred to as “ER agonists”. Those which, when given in combination with 17β-estradiol, block its effects are called “ER antagonists”.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist which sterically hinders the protein sequences required for receptor-coregulatory protein interaction [Pike, A. C. W., EMBO 1999, 18: 4608-4616; Shiau, A. K., Cell 1998, 95: 927-937].

As mentioned above, ER ligands have been historically classified as ER agonists or antagonists. In reality there is a continuum between these activities and indeed some compounds behave as estrogen receptor agonists in some tissues and estrogen receptor antagonists in others. The precise reason why the same compound can have cell-specific effects has not been elucidated, but the differences in receptor conformation and/or in the milieu of coregulatory proteins have been suggested.

Tamoxifen, a case in point, was initially developed as an ER antagonist for breast cancer treatment. It was subsequently discovered that, while an ER antagonist in the breast, it had ER agonist activity in the bone and uterus [Jordan, C. V., Breast Cancer Res 1987, 4: 31-35]. This unexpected finding of mixed ER agonist and antagonist activity within a single compound spawned efforts to develop other selective compounds with improved profiles, and now tamoxifen is referred to as a “first generation SERM” or “tissue selective estrogen” [McDonnell, D. P., J Soc Gyn Invest 2000, 7: S10-S15; Goldstein, S. R., Human Reproduction Update 2000, 6:212-224].

The development of raloxifene (a second generation SERM), originally aimed at the breast cancer treatment market to compete with tamoxifen, was redirected toward the treatment and prevention of osteoporosis in postmenopausal women. Preclinical data revealed that it spared bone, lowered LDL cholesterol, while demonstrating minimal estrogenic impact on the uterus and mammary gland. Other SERMs including bazedoxifene are currently in development.

### DESCRIPTION OF THE INVENTION

This invention provides extended dosing regimens (less frequently than daily) for the SERM bazedoxefine (1-[4-(2-azepan-1-yl-ethoxy)-benzyl]-2-(4-hydroxy-phenyl)-3-methyl-1H-indol-5-ol), or a pharmaceutically acceptable salt or prodrug thereof, particularly bazedoxifene acetate, which range from administering bazedoxifene every second day to administering bazedoxifene once every week.

As used herein, the term “extended dosing regimen” means administration ranging from every second day to once weekly.

As used herein, unless otherwise modified by a specific salt or prodrug, the term “bazedoxifene” means bazedoxifene, its pharmaceutically acceptable salts and prodrugs.

The preparation of bazedoxifene and its pharmaceutically acceptable salts is described in U.S. Pat. No. 5,998,402, which is hereby incorporated by reference. Bazedoxifene acetate has been evaluated in clinical trials using dosages of 10, 20, and 40 mg/day. Based on the results of these trials, it is anticipated that doses between 5 and 80 mg/day will provide satisfactory results.

The ability of bazedoxifene to be successfully administered on an extended dosing regimen was established in an in vivo standard pharmacological test procedure which evaluated bone mineral density (BMD), change in body weight, and uterine weight using bazedoxifene acetate as a representative compound of this invention. The following briefly describes the procedure used and results obtained in the standard pharmacological test procedure.

Female Sprague Dawley CD rats, ovariectomized (ovx) or sham ovx, were obtained 1 day after surgery from Taconic Farm (weight range 240-275 g). They were housed 3 or 4 rats/cage in a room on a 14/10 (light/dark) schedule and provided with food (Purina 500 rat chow) and water ad libitum. Treatment for all studies began 1 day after the animals arrival and dosed either daily or once weekly as indicated for 6 weeks. A group of age matched sham operated rats not receiving any treatment served as an intact, estrogen replete control group for each study. All treatments were prepared in 1% tween 80 in normal saline at defined concentrations so that the treatment volume was 0.1 mL/100 g body weight.

Five weeks after the initiation of treatment and one week prior to the termination of the study, each rat was evaluated for bone mineral density (BMD). The BMD's of the proximal tibiae (PT) and fourth lumbar vertabrae (L4) were measured in anesthetized rats using a dual energy X-ray absorptiometer (Eclipse XR-26, Norland Corp. Ft. Atkins, Wis.). The dual energy X-ray absorptiometer (DXA) measurements for each rat were performed as follows: Fifteen minutes prior to DXA measurements, the rat was anesthetized with an intraperitoneal injection of 100 mg/kg ketamine (Bristol Laboratories, Syracuse, N.Y.) and 0.75 mg/kg acepromazine (Aveco, Ft.Dodge, Iowa). The rat was placed on an acrylic table under the DXA scanner perpendicular to its path; the limbs were extended and secured with paper tape to the surface of the table. A preliminary scan was performed at a scan speed of 50 mm/second with a scan resolution of 1.5 mm×1.5 mm to determine the region of interest in PT and L4. Small subject software was employed at a scan speed of 10 mm/second with resolution of 0.5 mm X 0.5 mm for final BMD measurements. The software allows the operator to define a 1.5 cm wide area to cover the total length of L4. The BMDs for respective sites were computed by the software as a function of the attenuation of the dual beam (46.8 KeV and 80 KeV) X-ray generated by the source underneath the subject and the detector travelling along the defined area above the subject. The data for BMD values (expressed in g/cm2) and individual scans were stored for statistical analysis.

One week after BMD evaluation the rats were euthanized by carbon dioxide suffocation. The uteri were removed and the weights taken. The following table summarizes the results that were obtained. The sham group was not ovariectomized; all other groups were ovariectomized. In the table below, bazedoxifene acetate is abbreviated as BZA.

| | | | Trabecular | Δ Body | |
|---|---|---|---|---|---|
| Treatment | N | Total BMD^{a} | BMD^{a} | Weight^{b} | Uterine Weight^{c} |
| Sham | 8 | 681.96 ± 10.5* | 552.71 ± 16.8* | 47.4 ± 4.6* | 594.0 ± 73.0* |
| Vehicle | 8 | 550.51 ± 20.8 | 359.93 ± 27.8 | 91.6 ± 9.3 | 99.7 ± 5.1 |
| BZA 0.3 mg/kg | 8 | 604.33 ± 18.6* | 425.90 ± 26.3* | 60 ± 5.6* | 140.3 ± 6.3 |
| 7×/week | | | | | |
| BZA 0.3 mg/kg | 8 | 556.56 ± 20.5 | 361.93 ± 34.5 | 80.5 ± 2.0 | 131.3 ± 7.0 |
| 1×/week | | | | | |
| BZA 1.0 mg/kg | 8 | 562.90 ± 7.7 | 353.03 ± 15.7 | 71.3 ± 4.8* | 149.9 ± 5.0 |
| 1×/week | | | | | |
| BZA 3.0 mg/kg | 8 | 589.36 ± 10.9 | 427.00 ± 10.0* | 59.3 ± 7.0* | 127.3 ± 10.9 |
| 1×/week | | | | | |

^{a}Mean(mg/cm³) ± SEM
^{b}Mean(g) ± SEM
^{c}Mean(mg) ± SEM
*p < 005 vs. corresponding vehicle

The results obtained in the standard pharmacological test procedure using bazedoxifene acetate as a representative compound, demonstrate that bazedoxifene can be administered on an extended dosing regimen, while retaining efficacious results. Based on the results shown above, bazedoxifene can be administered according to an extended dosing regimen ranging from once every two days, to once per week. The dosage for a given dosing regimen can be given all at once or given multiple times on the same day. Based on individual patient needs, bazedoxifene can be aministered every second day, every third day, every fourth day, every fifth day, every sixth day, or every seventh day (once weekly). The administration period can also be adjusted depending on the needs of the patient, and still be considered to be administered according to an extended dosing regimen. For example, the dosage can be given once every other day, and then after medical follow-up be adjusted to be administered every third day, and eventually once weekly. When administered in this fashion, it is still conisdered to be administered according to an exended dosing regimen. It is preferred that the extended dosing regimen be administration once weekly, where the weekly dosage is given on one day, either as a single dose, or divided into two or more doses during the same day. It is preferred that the oral daily dosage in humans is between 5-80 mg. When bazedoxifene is administered once weekly, it is preferred that the once per week dosage will be from 3-15 times that of the daily dosage. Accordingly, it is preferred that the once weekly oral dosage be between 15 and 1200 mg given once per week; with the dosage being given in one or more doses during the administration day. When bazedoxifene is administered according to an every second day regimen, it is preferred that the daiy dosage be from the daily dosage to 5 times that of the daily dosage. Accordingly, it is preferred in the once every second day regimen, that the oral dosage be between 5 and 400 mg given once every second day, with the dosage being given in one or more doses during the administration day. It is preferred that the extended dosage period will be once weekly administration.

The uses of bazedoxifene are disclosed in U.S. Pat. No. 5,998,402, which is hereby incorporated by reference. Such uses include owering cholesterol cholesterol, triglycerides, Lp(a), or LDL levels; inhibiting or treating hypercholesteremia; or hyperlipidemia. Bazedoxifene is useful in the treatment and inhibition of bone loss, which may result from an imbalance in a individual's formation of new bone tissues and the resorption of older tissues, leading to a net loss of bone. Such bone depletion results in a range of individuals, particularly in post-menopausal women, women who have undergone bilateral oophorectomy, those receiving or who have received extended corticosteroid therapies, those experiencing gonadal dysgenesis, and those suffering from Cushing's syndrome. Special needs for bone, including teeth and oral bone, replacement can also be addressed using these compounds in individuals with bone fractures, defective bone structures, and those receiving bone-related surgeries and/or the implantation of prosthesis. In addition to those problems described above, bazedoxifene can be used in treatments for osteoarthritis, hypocalcemia, hypercalcemia, Paget's disease, osteomalacia, osteohalisteresis, multiple myeloma and other forms of cancer having deleterious effects on bone tissues.

Bazedoxifene is also useful for treating many maladies which result from estrogen effects and estrogen excess or deficiency including osteoporosis, prostatic hypertrophy, male pattern baldness, vaginal and skin atrophy, acne, dysfunctional uterine bleeding, endometrial polyps, benign breast disease, uterine leiomyomas, adenomyosis, ovarian cancer, infertility, breast cancer, endometriosis, endometrial cancer, polycystic ovary syndrome, cardiovascular disease, contraception, Alzheimer's disease, cognitive decline and other CNS disorders, as well as certain cancers including melanoma, prostate cancer, cancers of the colon, CNS cancers, among others. Additionally, bazedoxifene can be used for contraception in pre-menopausal women, as well as hormone replacement therapy in post-menopausal women or in other estrogen deficiency states where estrogen supplementation would be beneficial. Bazedoxifene may also be used in disease states where amenorrhea is advantageous, such as leukemia, endometrial ablations, chronic renal or hepatic disease or coagulation diseases or disorders.

It is preferred that bazedoxifene is administered orally. Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, , xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations herein may utilize standard delay or time release formulations to alter the absorption of the active compound(s). Suppository formulations may be made from traditional materials, including cocoa butter, with or without the addition of waxes to alter the suppository's melting point, and glycerin. Water soluble suppository bases, such as polyethylene glycols of various molecular weights, may also be used.

Solid oral formulations, preferably in the form of a film coated tablet or capsule, useful for this invention include the active pharmacological agents disclosed herein in combination with carrier or excipient systems having the components:

a) a filler and disintegrant component comprising from about 5% to about 82% by weight (wght) of the total formulation, preferably between about 30% and about 80% of the formulation, of which from about 4% to about 40% by weight of the total formulation comprises one or more pharmaceutically acceptable disintegrants;

b) optionally, a wetting agent comprising from about 0.2 to about 5% of the composition (wght), such as selected from the group of sodium lauryl sulfate, polyoxyethylene sorbitan fatty acid esters, polyoxyethylene alkyl ethers, sorbitan fatty acid esters, polyethylene glycols, polyoxyethylene castor oil derivatives, docusate sodium, quaternary ammonium compounds, sugar esters of fatty acids and glycerides of fatty acids;

c) a lubricant comprising from about 0.2% to about 10% of the composition (wght), such as selected from the group of magnesium stearate or other metallic stearates (e.g. calcium stearate or zinc stearate), fatty acid esters (e.g. sodium stearyl fumarate), fatty acids (e.g. stearic acid), fatty alcohols, glyceryl behenate, mineral oil, parrafins, hydrogenated vegetable oils, leucine, polyethylene glycols, metallic lauryl sulfates and sodium chloride; and

d) optionally, a glidant comprising from about 0.1% to about 10% (wght) of the composition, the glidant selected from those known in the art, including from the group of silicon dioxide, talc, metallic stearates, calcium silicate, or metallic lauryl sulfates.

While the formulations described herein may be used in an uncoated or non-encapsulated solid form, preferably the final compositions are coated or encapsulated. The pharmacological compositions may be optionally coated with a film coating, preferably comprising from about 0.3% to about 8% by weight of the overall composition. Film coatings useful with the present formulations are known in the art and generally consist of a polymer (usually a cellulosic type of polymer), a colorant and a plasticizer. Additional ingredients such as wetting agents, sugars, flavors, oils and lubricants may be included in film coating formulations to impart certain characteristics to the film coat. The compositions and formulations herein may also be combined and processed as a solid, then placed in a capsule form, such as a gelatin capsule.

The filler component listed above may utilize the filler or binder components known in the art for solid oral formulations. Pharmaceutically acceptable fillers or binding agents selected from those known in the art including, but not limited to, lactose, microcrystalline cellulose, sucrose, mannitol, calcium phosphate, calcium carbonate, powdered cellulose, maltodextrin, sorbitol, starch, or xylitol.

In conjunction with or in place of the materials listed above for the filler component, the present formulations utilize disintegrant agents. These disintegrants may be selected from those known in the art, including pregelatinized starch and sodium starch glycolate. Other useful disintegrants include croscarmellose sodium, crospovidone, starch, alginic acid, sodium alginate, clays (e.g. veegum or xanthan gum), cellulose floc, ion exchange resins, or effervescent systems, such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.). The disintegrant(s) useful herein will comprise from about 4% to about 40% of the composition by weight, preferably from about 15% to about 35%, more preferably from about 20% to about 35%. Some components may have multiple functions in the formulations of this invention, acting e.g. as both a filler and a disintegrant, such a component may be referred to as a filler disintegrant and its function in a specific formulation may be singular even though its properties may allow niultiple functionality.

The pharmaceutical formulations and carrier or excipient systems herein preferably also contain an antioxidant or a mixture of antioxidants, most preferably ascorbic acid. Other antioxidants which may be used include sodium ascorbate and ascorbyl palmitate, preferably in conjunction with an amount of ascorbic acid. A preferable range for the antioxidant(s) is from about 0.5% to about 15% by weight, most preferably from about 0.5% to about 5% by weight.

Among the formulations of this invention are pharmaceutical formulations containing a pharmaceutically effective amount of an active pharmacological agent and a carrier or excipient system comprising:

a) a filler and disintegrant component comprising between about 50% and about 87% of the formulation, with from about 4% to about 40% of the formulation comprising one or more disintegrant agents;

b) a wetting agent comprising between about 0.5% and about 2.7% of the formulation;

c) a lubricant comprising between about 0.2% and about 5.5% of the formulation; and

d) a glidant comprising between about 0.1% and about 5.5% of the formulation.

The percentages listed in the formulations above indicate percentages by weight of the total weight of the components listed from a) to d). The formulations above also preferably contain an optional antioxidant component, preferably ascorbic acid, at a concentration of from about 0.5% to about 5.5% by weight of the formulation. The formulations are also preferably contained within a pharmaceutically acceptable capsule, such as a gel capsule, or coated with a film coating comprising from about 0.3% to about 8% by weight of the formulation.

This invention also comprises a pharmaceutical carrier or excipient systems useful in pharmaceutical compositions utilizing as an active ingredient one or more of the compounds described herein, or a pharmaceutically acceptable salt thereof, as described herein. These pharmaceutical carrier or excipient systems comprise, by weight:

a) a filler and disintegrant component comprising between about 54% and about 80% of the formulation, with the disintegrant agent(s) therein comprising from about 4% to about 40% by weight of the overall formulation;

b) a wetting agent comprising between about 0.55% and about 2.5% of the formulation;

c) a lubricant comprising between about 0.2% and about 5.5% of the formulation; and

d) a glidant comprising between about 0.1% and about 5.0% of the formulation.

The more preferred carrier or excipient systems above also optionally and preferably contain an antioxidant component, preferably ascorbic acid, at a concentration of from about 0.1% to about 5.0% by weight.

Among the carrier or excipient systems of this invention are those comprising:

a) a filler and disintegrant component, as described above, comprising between about 50% and about 87% of the formulation, the disintegrant(s) therein comprising from about 25% to about 35% of the formulation, by weight;

b) a wetting agent comprising between about 0.55% and about 2.7% of the formulation;

c) a lubricant comprising between about 0.2% and about 5.5% of the formulation;

d) a glidant comprising between about 0.1% and about 5.5% of the formulation; and

e) an antioxidant component, preferably ascorbic acid, at a concentration of from about 0.1% to about 5.5% by weight.

### EXAMPLE 1

### Bazedoxifene Acetate-Rapid Dissolution Formulations

| | without | With |
|---|---|---|
| | Ascorbic | Ascorbic |
| Ingredient | Acid | Acid |
| Bazedoxifene acetate, | 10.00 | 10.00 |
| micronized* | | |
| Lactose NF fast flow | 33.10 | 31.60 |
| Microcrystalline | 25.00 | 25.00 |
| Cellulose, NF (Avicel | | |
| PH101) | | |
| Starch 1500 | 20.00 | 20.00 |
| Sodium Lauryl Sulfate | 1.50 | 1.50 |
| NF | | |
| Sodium Starch Glycolate | 10.00 | 10.00 |
| Ascorbic Acid USP | — | 1.5 |
| Syloid 244 FP | 0.15 | 0.15 |
| Magnesium Stearate | 0.25 | 0.25 |

*Amount in formula is adjusted for actual potency of bazedoxifene as free base. Corresponding adjustment made with lactose.

The formulations given above in Table 1 were prepared by incorporating a portion of the excipients in the granulation and a portion is also added in the final blending steps as dry powders. A dissolution profile generated for the formulations demonstrated almost 90% release of the drug in 30 minutes. Thus, the unique combination of disintegrants and soluble diluents plus the incorporation of both granulated and powdered solids into the composition ensures the fastest release of drug.

Wet granulation of the formulations as described in Table 1 may be carried out by mixing the drug and ascorbic acid with a portion of the lactose, microcrystalline cellulose, pregelatinized starch and sodium starch glycolate. The sodium lauryl sulfate is dissolved in the water and used to granulate the mixture of powders in a high shear mixer. The granulation is dried in a fluid bed dryer to a moisture of 2-3%. The particle size of the dried granulation is controlled by passing through a mill equipped with knife-edged blades and using a 20- or 30-mesh screen. The silicon dioxide and remaining lactose, microcrystalline cellulose, pregelatinized starch, and sodium starch glycolate are mixed with the milled granulation in a tumble-type mixer. The final blend is prepared by adding magnesium stearate to the tumble-type mixer and mixing. Compression is carried out on a rotary tablet press using appropriate size tooling. Coating is performed in conventional coating pans and applying the coating suspension to achieve a suitable film coat.

### EXAMPLE 2

### Modified Bazedoxifene Acetate Formulation % w/w

| | 5% |
|---|---|
| Ingredient | granulation |
| Bazedoxifene acetate, micronized^{a} | 5.00 |
| Lactose NF | 41.00 |
| Microcrystalline Cellulose, NF | 35.00 |
| Pregelatinized Starch NF | 10.00 |
| Sodium Lauryl Sulfate NF | 1.50 |
| I-Ascorbic Acid USP | 1.50 |
| Sodium Starch Glycolate NF | 5.50 |
| Magnesium Stearate NF | 0.50 |
| Pur. Water USP^{b} | qs |

^{a}Amount in formula is adjusted for actual potency of bazedoxifene acetate as free base. Corresponding adjustment made with Lactose.
^{b}Used in process but does not appear in the final product.

### EXAMPLE 3

### Bazedoxifene Acetate at 5% Granulation

A preferred carrier or excipient system for formulating a granulation of from about 2 to about 8% by weight of one of the active pharmacological agents of this invention, preferably about 5%, may be produced utilizing the carrier or excipient components on a weight percentage; lactose from about 32% to about 38%, microcrystalline cellulose from about 32% to about 38%, pregelatinized starch from about 12% to about 16%, ascorbic acid from about 1% to about 2%, sodium lauryl sulfate from about 1% to about 2%, sodium starch glycolate from about 4% to about 8%, silicon dioxide from about 0.1% to about 0.2% and magnesium stearate from about 0.3% to about 0.7%.

A formulation of this invention utilizing bazedoxifene as the active ingredient at a 5% granulation was prepared utilizing the components listed below in a granulation part of components and a dry part.

| Item No. | Ingredients | Mg/Unit |
|---|---|---|
| Granulation Part: | | |
| 1 | Bazedoxifene acetate | 5.00 |
| 2 | Lactose NF | 26.60 |
| 3 | Microcrystalline Cellulose NF | 25.00 |
| 4 | Pregelatinized Starch NF | 10.00 |
| 5 | Ascorbic Acid USP | 1.50 |
| 6 | Sodium Lauryl Sulfate NF | 1.50 |
| 7 | Sodium Starch Glycolate NF | 4.00 |
| 8 | Water, Purified USP | Q.S. |
| | | 73.60 |
| Dry Part: | | |
| 12 | Lactose NF (fast flo) | 9.75 |
| 10 | Microcrystalline Cellulose NF | 10.00 |
| 11 | Pregelatinized Starch NF | 4.00 |
| 12 | Sodium Starch Glycolate NF | 2.00 |
| 13 | Silicon Dioxide NF | 0.15 |
| 14 | Magnesium Stearate NF | 0.50 |
| | | 100.00 |

A film coat of White Opadry I (YS-1-18027-A) was applied to the tablets, which were compressed as follows:

| Dose of Bazedoxifene | tablet weight, mg | mg of film coat applied/tablet |
|---|---|---|
| 5 mg | 100 | 6.0 |
| 10 mg | 200 | 8.0 |
| 20 mg | 400 | 13.0 |

It is intended that each of the patents, applications, and printed publications including books mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A method of treating or inhibiting bone loss in a mammal in need thereof, which comprises administering an effective amount of bazedoxifene according to an extended dosing regimen.

2. The method according to claim 1, wherein the bazedoxifene is bazedoxifene acetate.

3. The method according to claim 2, wherein the bazedoxifene is administered once weekly

4. The method according to claim 2, wherein the bazedoxifene is administered every second day.

5. A method of lowering cholesterol, triglycerides, Lp(a), or LDL levels; inhibiting or treating hypercholesteremia; or hyperlipidemia; in a mammal in need thereof, which comprises administering an effective amount of bazedoxifene according to an extended dosing regimen.

6. The method according to claim 5, wherein the bazedoxifene is bazedoxifene acetate.

7. The method according to claim 6, wherein the bazedoxifene is administered once weekly

8. The method according to claim 6, wherein the bazedoxifene is administered every second day.

9. A method of treating or inhibiting cardiovascular disease in a mammal in need thereof, which comprises administering an effective amount of bazedoxifene according to an extended dosing regimen.

10. The method according to claim 9, wherein the bazedoxifene is bazedoxifene acetate.

11. The method according to claim 10, wherein the bazedoxifene is administered once weekly

12. The method according to claim 10, wherein the bazedoxifene is administered every second day.

13. A method of treating or inhibiting dementias, neurodegenerative disorders, and Alzheimer's disease; providing neuroprotection or cognition enhancement in a mammal in need thereof, which comprises administering an effective amount of bazedoxifene according to an extended dosing regimen.

14. The method according to claim 13, wherein the bazedoxifene is bazedoxifene acetate.

15. The method according to claim 14, wherein the bazedoxifene is administered once weekly

16. The method according to claim 14, wherein the bazedoxifene is administered every second day.

17. A method of treating or inhibiting osteoporosis in a mammal in need thereof, which comprises administering an effective amount of bazedoxifene according to an extended dosing regimen.

18. The method according to claim 17, wherein the bazedoxifene is bazedoxifene acetate.

19. The method according to claim 18, wherein the bazedoxifene is administered once weekly

20. The method according to claim 18, wherein the bazedoxifene is administered every second day.
